(19) United States
(12) Patent Application Publication
(10) Pub. No.: US 2006/0073167 A1
(43) Pub. Date: Apr. 6, 2006

(54) Vaccine for fish cold-water disease

(75) Inventors: Syunichirou Oshima, Kochi (JP); Motoki Kondo, Kochi (JP); Kenji Kawai, Kochi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: TECHNO NETWORK SHIKOKU LTD., Kagawa (JP)

(21) Appl. No.: 10/538,882

(22) PCT Filed: Dec. 17, 2003
(86) PCT No.: PCT/JP03/16180

§ 371 Date: Jun. 14, 2005

(30) Foreign Application Priority Data
Dec. 18, 2002 (JP) 2002-366769

(51) Int. Cl. A61K 39/02

(52) U.S. Cl. 424/234.1

(57) ABSTRACT

The present invention provides a vaccine for the bacterial cold-water disease in fish comprising inactivated cells of Flavobacterium psychrophilium in the logarithmic growth phase or components thereo

### FIELD OF THE INVENTION

The present invention relates to a vaccine against (bacterial) cold-water disease in fishes and a method for preventing the disease in fish using the vaccine.

### BACKGROUND OF THE INVENTION

Cold-water disease is a disease occurring in salmon, trout, ayu (sweetfish) and crucian carp in low water temperature seasons. This disease, which attacks young fish in low water temperature seasons and has a high mortality, was originally discovered in trout in North America. While the mortality rate is 20 to 50%, another problem is that sequelae such as ulcers remain on the surface of the fish that have escaped death.

Although therapy for cold-water disease include raising the water temperature or oral administration of sodium sulfizole, raising the water temperature above 25° C. is uneconomical treatment while administration of drugs is not preferable for edible fish.

It has been proved that the pathogen of the cold-water disease is Flavobacterium psychrophilium, which is also known as Flexibactor cyclophils or Cytophagar cyclophils. However, no vaccines against this disease have yet been developed.

### SUMMARY OF THE INVENTION

Accordingly, it is an object of the present invention to provide the vaccine against cold-water disease in fish.

The inventors of the present invention have investigated Flavobacterium psychrophilium as a pathogen of the cold-water disease in terms of pathogenicity and vaccine activity depending on various cultivation conditions, and found a quite unexpectedly that the vaccine activity becomes higher by using bacteria in a logarithmic growth phase rather than by using bacteria in a stationary-state phase. The present invention has been completed based on this findings.

In a first aspect, the present invention provides the vaccine for cold-water disease in fish comprising inactivated cells of Flavobacterium psychrophilium in a logarithmic growth phase or components of the cells.

In a second aspect, the present invention provides the vaccine composition for cold-water disease in fish containing inactivated cells of Flavobacterium psychrophilium in a logarithmic growth phase or components of the cells.

In a third aspect, the present invention provides the method for preventing cold-water disease in fish comprising administering an effective dosage of inactivated cells of Flavobacterium psychrophilium in a logarithmic growth phase or components of the cells.

It may be concluded that cold-water disease of salmon, trout, carp and ayu can be efficiently prevented by using the vaccine of the present invention.

### BRIEF DESCRIPTION OF THE DRAWINGS

FIG. 1 is a graph showing the relationship between the culture time and optical density (OD) at 600 nm and the number of cells (CFU/mL);

FIG. 2 is a graph showing the pathogenicity (accumulated mortality) of ayu depending on the culture conditions of the bacteria of the present invention;

FIG. 3 shows the results of the SDS-PAGE analysis of the cell components of the bacteria of the present invention;

FIG. 4 shows scanning electron microscope photographs (A, C and E=×20,000 magnification; B, D and F=×100,000 magnification) of the bacteria of the present invention at logarithmic growth phases (A and B: 36 hours) and at stationary phases (C and D: 48 hours, E and F: 72 hours);

FIG. 5 shows transmission electron microscope photographs of ultra-thin slices of the bacteria of the present invention in the logarithmic growth phase.

FIG. 6 shows scanning electron microscope photographs of the lower jaw of ayu infected with the bacteria of the present invention;

FIG. 7 shows the survival rate in challenge 1 (challenge 3 weeks after administration of the vaccine);

FIG. 8 shows the survival rate in challenge 2 (challenge 7 weeks after administration of the vaccine);

FIG. 9 is a photograph showing the symptoms of the dead ayu (the arrows show the symptoms specific to the cold-water disease); and

FIG. 10 is a photograph showing the results of diagnosis of infection, if any, of dead ayu with the bacteria of the present invention detected by a fluorescent antibody test;

FIG. 11 is a graph showing the pathogenicity (accumulated mortality) of the bacteria of the present invention against rainbow trout; and

FIG. 12 shows photographs of healthy rainbow trout in the control group (A), symptoms of dead rainbow trout that died on day 1 after challenge by immersion (B, C and D), symptoms of dead rainbow trout that died on day 5 after challenge by immersion (E and F), and Flavobacterium psychrophilium found in the caudal fins of dead rainbow trout.

### BEST MODE FOR CARRYING OUT THE INVENTION

Inactivated cells of Flavobacterium psychrophilium (may be referred to as the bacteria of the present invention hereinafter) in a logarithmic growth phase or components of the cells are used in the vaccine of the present invention. Usually, bacterial cultivation phases can be divided into a lag phase, logarithmic growth phase, stationary phase, extinction phase and survival phase. Many projections were observed on the surface of invading bacterial cells upon observation of the bacterial cells of the present invention invading fish bodies. On the other hand, differences of cell secretory products were detected by SDS-PAGE and the existence of projections was observed on the surface of the bacterial cells in the logarithmic growth phase upon observation of the configuration and analysis of the bacteria of the present invention in the lag phase, logarithmic growth phase and stationary phase.

The bacterial cells of the present invention used for production of the vaccine are obtained by cultivating the cells according to conventional methods and by harvesting the cells in the logarithmic growth phase. The bacterial cells of the present invention may be inoculated on an appropriate culture medium and cultivated according to conventional methods. The culture medium preferably contains an appropriate amount of assimilable carbon and nitrogen sources.

The carbon and nitrogen sources are not particularly restricted. Examples of them include tripton, serum of various animals, corn gluten meal, soy bean powder, corn steep liquor, casamino acid, yeast extract, pharma media, sardine meal, meat extract, peptone, HiPro®, AjiPower®, corn meal, soy bean meal, coffee refuse, cotton seed oil refuse, Cultivator®, Amiflex® and Ajipron®, Zest® and Ajix®. Examples of the carbon source include assimilable carbon sources such as arabinose, xylose, glucose, mannose, sucrose, maltose, soluble starch, lactose and cane molasses, and assimilable organic acids such as acetic acid. Phosphates, organic salts such as Mg²⁺, Ca²⁺, Mn²⁺, Zn²⁺, Co²⁺, Na⁺ and K⁺ salts, and other inorganic salts and trace amounts of nutrients, if necessary, may also be added to the culture medium. Commercially available culture media such as TY culture medium and Cytophagar (CYT) culture medium, as well as modified Cytophaga (MCYT) culture medium and culture medium supplemented with bovine fetal serum may also be used.

The culture condition is preferably controlled at pH 6.8 to 8.4 and at a temperature of 4 to 20° C.

Whether the bacteria of the present invention are in the logarithmic growth phase or not may be confirmed by measuring the optical density at 600 nm, which dramatically increases in the logarithmic growth phase. For example, cultivation reaches the logarithmic growth phase after 20 to 30 hours' cultivation at pH 7.3 and 15° C.

The bacteria of the present invention in the logarithmic growth phase are separated by centrifugation or filtration, or the culture product may be directly inactivated. The inactivation treatment includes heat treatment or formalin treatment.

The bacteria of the present invention contain cell membrane components, vesicles and secretary products. These components are preferably collected after ultrasonic pulverization of the inactivated bacterial cells.

The inactivated bacterial cells and components thereof are preferably used after filtration, or after concentration by evaporation or lyophilization.

Although the inactivated bacterial cells of the present invention may be directly used as the vaccine, they may be formulated into a vaccine composition together with a pharmaceutically acceptable liquid or solid carrier. Examples of the formulation of the vaccine composition include oral administration compositions, injection compositions, compositions for immersing fish, and feed compositions. Examples of the liquid carrier include water and physiological saline, while examples of the solid carrier include excipients such as talc and sucrose. The inactivated bacterial cells of the present invention or components thereof may be mixed with conventional fish feeds to prepare the feed composition. An adjuvant may be added to these vaccine compositions in order to enhance the antigenicity.

While the vaccine or vaccine composition of the present invention may be administered to adult fish, it is preferably administered before the onset of cold-water disease, for example, during the period when the fish is young. The dosage is preferably about 1 mg to 5 g per 1 kg of the body weight as converted into the weight of the inactivated bacterial cells or components thereof. The dosage may be once or several times, for example 2 to 10 times. The vaccine may be administered every day, or with an interval of 1 to 2 days.

The fish that can be administered the vaccine or vaccine composition of the present invention are not particularly restricted so long as the fish are afflicted by cold-water disease caused by the bacteria of the present invention; examples of the fishes include ayu (sweetfish) and crucian carp, and salmon and trout such as yamame (salmo masau), rainbow trout and silver trout.

### EXAMPLES

While the present invention is described in more detail hereinafter with reference to examples, the present invention is by no means restricted to these examples.

### Example 1

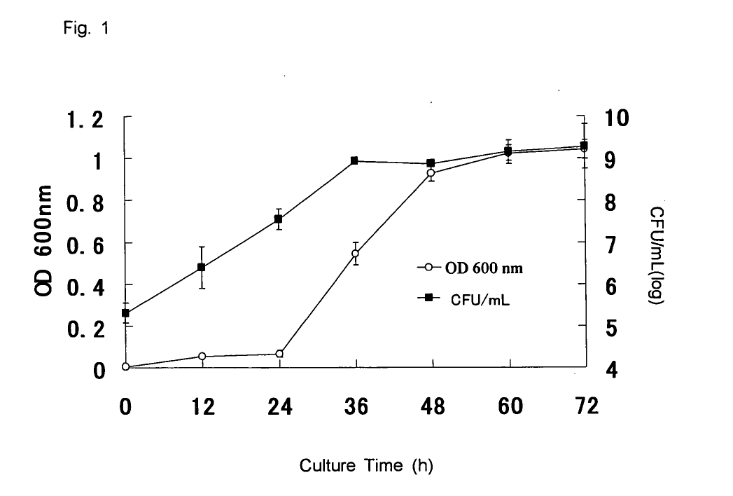

(1) Cells of Flavobacterium psychrophilium G3724 (this strain was used in the experiments hereinafter) contained in a platinum loop were inoculated on a 4-mL MCYT culture medium (trypton 2.0 g, yeast extract 0.5 g, meat extract 0.2 g, sodium acetate 0.2 g, calcium chloride 0.2 g, distilled water 1000 mL, pH 7.2). After cultivation at 15° C. for 2 days, a 0.5-mL fraction of the culture medium was inoculated on a 200-mL MCYT culture medium followed by cultivation with shaking at 15° C. The relationship between the cultivation time, and the cell number and optical density at 600 nm is shown in FIG. 1. FIG. 1 shows that the lag phase is from 0 to 24 hours after inoculating, the logarithmic growth phase is 24 to 48 hours after inoculating, and the stationary phase is after 48 hours from inoculating in the bacteria of the present invention.

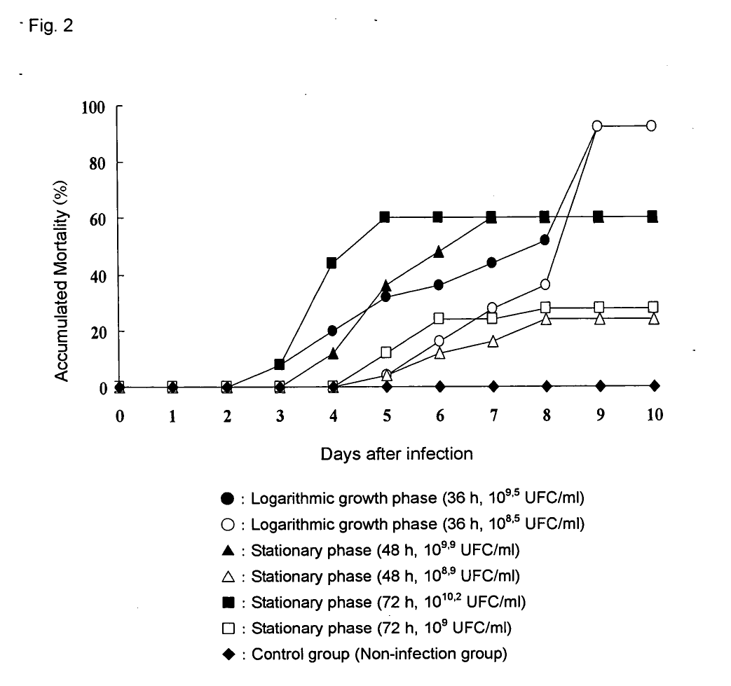

(2) The differences in pathogenicity of the bacteria of the present invention depending on the culture conditions were investigated. The bacteria of the present invention in the logarithmic growth phase and stationary phase were added to an aquarium of ayu at a concentration of 10⁸ to 10¹⁰ CFU/mL to determine the pathogenicity of the bacteria. Ayu used for the experiment had a body weight of 0.5 to 5 g, and the temperature of the aquarium was 15° C. As shown in FIG. 2, while the mortality rate of the fish in the infection group using the bacteria of the present invention in the stationary phase until day 10 of the experiment was 20 to 60% of the mortality rate of the fish in the control group (non-infection group), the mortality of the fish in the infection group using the bacteria of the present invention in the logarithmic growth phase at day 10 of the experiment was 100%, showing that the bacteria in the logarithmic growth phase have higher pathogenicity than the bacteria in the stationary phase.

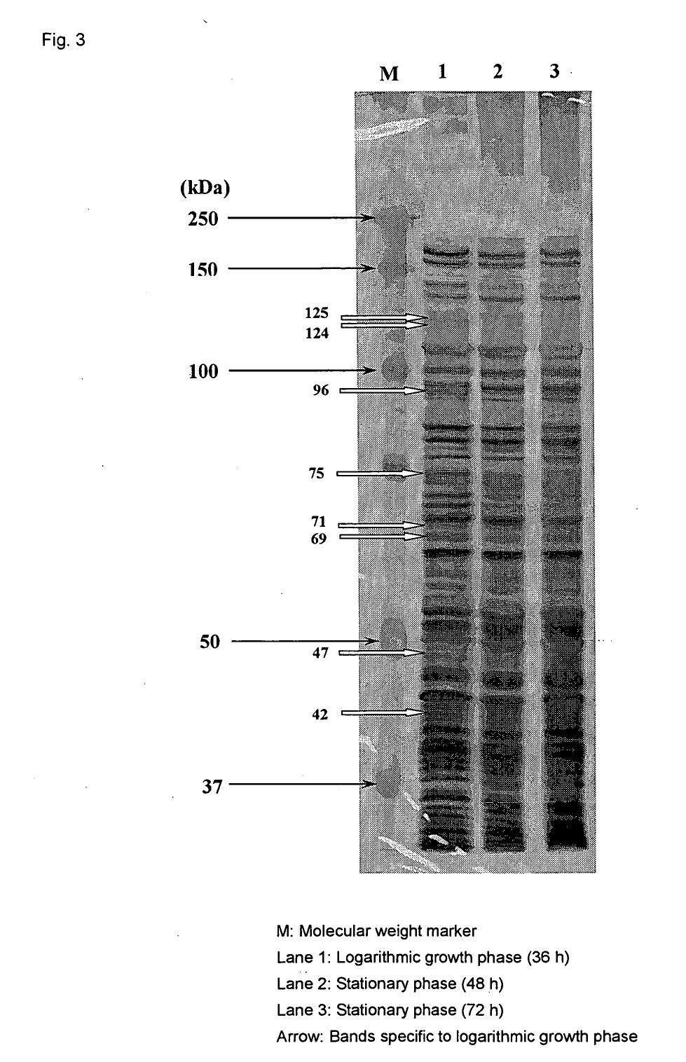

(3) The bacterial cells of the present invention in different growth phases were pulverized by ultrasonic waves. Each fraction of the extract was isolated by sodium dodecylsulfate-polyacrylamide gel electrophoresis (SDS-PAGE, silver staining). The results are shown in FIG. 3. The results show that certain substances are produced specifically in the logarithmic growth phase (indicated by arrows in the graph).

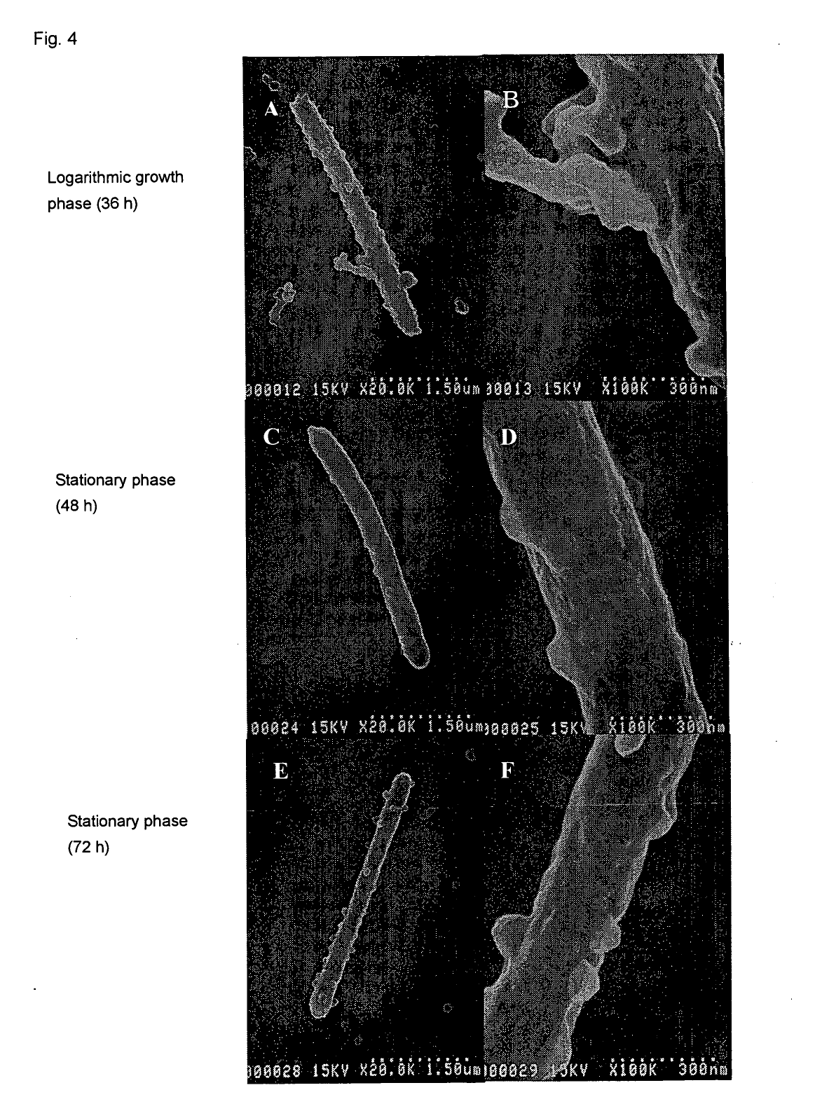

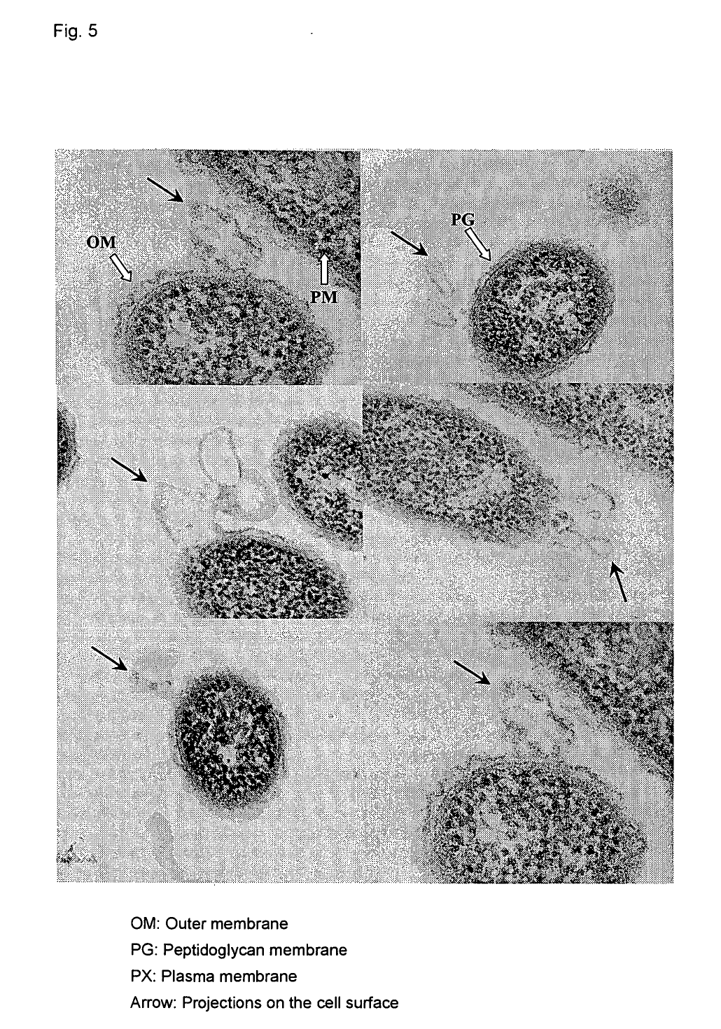

(4) The bacteria of the present invention in the logarithmic growth phase and stationary phase were observed under a scanning electron microscope (FIG. 4) and transmission electron microscope (FIG. 5). It was revealed from the results that projections can be seen on the surface of the bacterial cells in the logarithmic growth phase.

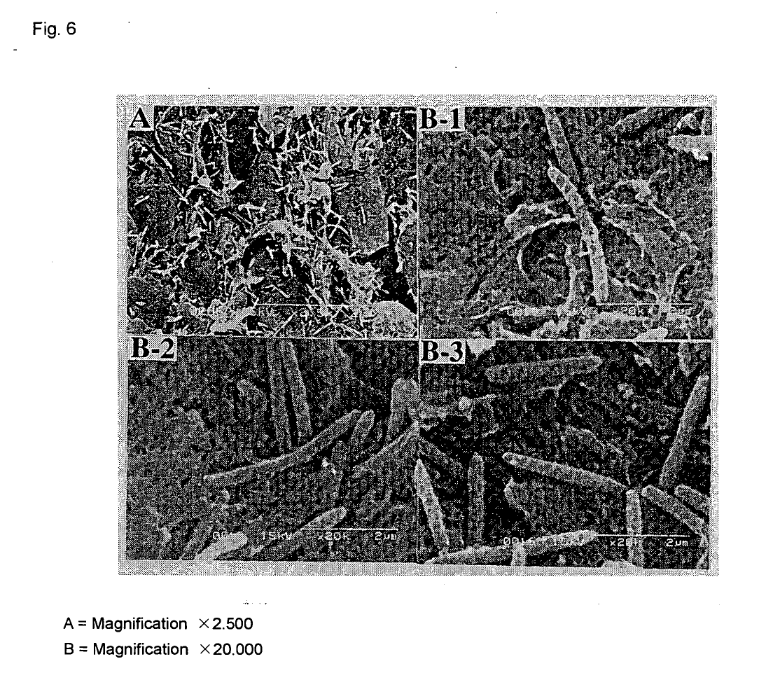

(5) Ayu were infected with the bacteria of the present invention in the logarithmic growth phase. It was observed under the scanning electron microscope that the bacteria of the present invention had invaded into the lower jaw of the ayu (FIG. 6). The result indicates that the bacteria of the present invention in the logarithmic growth phase having the vesicles invaded into the body of the ayu.

### Example 2

Flavobacterium psychrophilium G3724 was cultured in 1000 mL of the MCYT culture medium contained in a 2000-mL Sakaguchi flask at 15° C. The cells showing OD 0.2 to 0.7 at 600 nm were used as the bacterial cells in the logarithmic growth phase. Then, the cells as a culture product at a growth phase showing OD of 0.2 to 0.7 at 600 nm in the culture period of 24 to 36-hour were inactivated by incubation in 0.3% formalin at 15° C. for 2 days, and the inactivated bacterial cells were isolated by centrifugation at 4° C. and 8,000 to 10,000×g. The bacterial cells in the stationary phase after 36-hour cultivation (OD_{600 nm}=1.0) were also inactivated by the same method as described above to obtain inactivated bacterial cells as controls.

### Example 3

Cells of Flavobacterium psychrophilium G3724 contained in a platinum loop was inoculated on 50 mL of the MCYT culture medium and pre-cultured at 15° C. for 48 hours. A 2.5-mL fraction of this culture medium was inoculated on 1000 mL of the MCYT culture medium, followed by culture at 15° C. for 36 hours. OD at 600 nm was 02 to 0.7. The culture product was incubated in 0.3% formalin at 15° C. for 2 days. The bacterial cells were then collected by centrifugation at 8,000 to 10,000×g at 4° C. The cells obtained were re-suspended in physiological saline containing 0.3% formalin to obtain a vaccine suspension containing the inactivated bacterial cells of the present invention.

### Example 4

The inactivated bacterial cells obtained from the cells in the logarithmic growth phase and stationary phase in Example 2 were orally administered to ayu with an average body weight of 5.0 g at a dosage of 0.1 FKCg/kg/day.

After the oral administration as described above, the ayu were challenged by immersing in the bacterial solution. The results are shown in Table 1.

TABLE 1

| | Dosage of | | |
|---|---|---|---|
| | Challenge | Death/ | Survival |
| Group | (CFU/mL) | Challenge | Rate (%) |
| Logarithmic Growth Phase Group | 1.7 × 10⁸ | 39/152 | 74^{a,c} |
| Stationary Phase Group | 1.9 × 10⁸ | 39/105 | 63^{b} |
| Control Group | 2.2 × 10⁸ | 82/165 | 50 |

^{a}Significant difference against control group (p < 0.001), chi-square test
^{b}Significant difference against control group (p < 0.05)
^{c}Significant difference against stationary phase group (p < 0.05)

Table 1 shows that the difference in the survival rate was significant in both the stationary phase group and logarithmic growth phase group as compared with the control group. However, the survival rate of the logarithmic growth phase group was significantly higher than that of the stationary phase group, showing that the logarithmic growth phase group is particularly useful as the vaccine.

### Example 5

The effect of the vaccine was investigated using the vaccine composition obtained in Example 3. The vaccine was orally administered for 2 weeks (0.1 g/kg) to the fish from 5 weeks before the start of challenge, and the fish were fed on a standard feed for 3 weeks to enhance immunological activity. The fishes were then divided into two groups: one in which the challenge was started 3 weeks after the end of vaccine administration, and another in which the challenge was started 7 weeks after the end of vaccine administration.

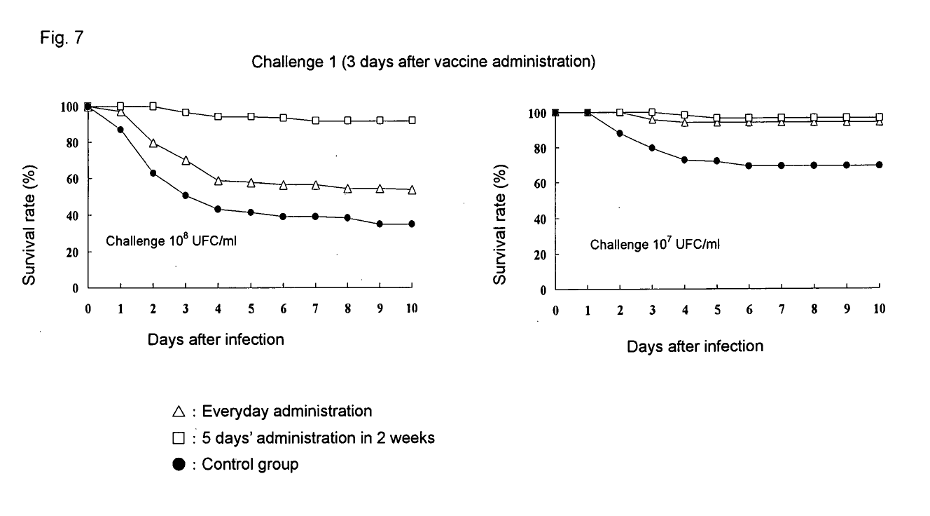

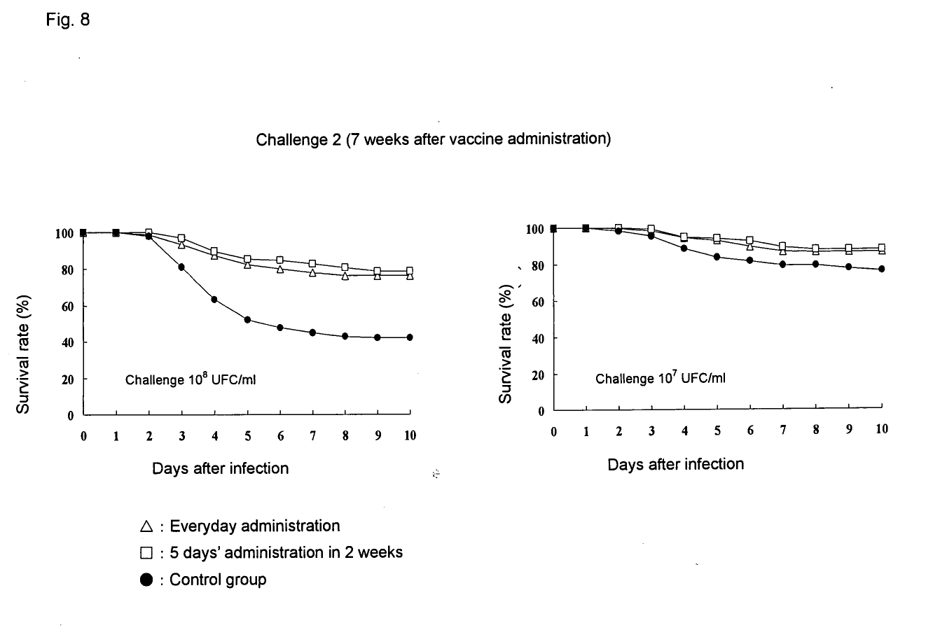

Two thousand “ayus” with a body weight of 0.5 g were divided into two groups. The vaccine was either orally administered every day to the fishes in one group, or five times in two weeks (oral administration with an interval of two days) to the fishes in the other group. The results are shown in Table 2, and in FIGS. 7 and 8.

TABLE 2

| | | | |
|---|---|---|---|
| Average | Amount of | No. of | |
| Body | Challenge | Deaths/No. of | Survival |
| Weight (g) | (CFU/mL) | Challenges | Rate (%) |

Challenge 1^{a}

| | | | | |
|---|---|---|---|---|
| 1 | 1.7 | | 7/118 | 94.1^{b} |
| 2 | 1.8 | 4.4 × 10⁷ | 4/119 | 96.6^{b} |
| Control | 1.8 | | 36/117 | 69.2 |
| 1 | 1.9 | | 53/114 | 53.5^{b} |
| 2 | 1.8 | 1.2 × 10⁸ | 10/120 | 91.7^{b} |
| Control | 1.9 | | 79/121 | 34.7 |

Challenge 2^{a}

| | | | | |
|---|---|---|---|---|
| 1 | 2.7 | | 26/186 | 86.6^{b} |
| 2 | 2.9 | 2.1 × 10⁷ | 20/168 | 88.1^{b} |
| Control | 2.7 | | 41/174 | 76.4 |
| 1 | 2.7 | | 40/170 | 76.5^{b} |
| 2 | 3.0 | 1.4 × 10⁸ | 36/165 | 78.8^{b} |
| Control | 3.2 | | 107/185 | 42.2 |

^{a}Challenge 1: challenged 3 weeks after administration of vaccine, Challenge 2: challenged 7 weeks after administration of vaccine
^{b}significant difference against control group (p < 0.01)
1: the group in which the vaccine was administered every day for 2 weeks
2: the group in which the vaccine was administered 5 times in 2 weeks

The results of the challenge tests three weeks after the administration of the vaccine show that a significant difference was observed between the vaccine-administered group and control group. It was also shown that the effect of the vaccine is higher in the group in which the vaccine was administered only five times than in the group in which the vaccine was administered every day.

The effect of the vaccine was significantly higher in both vaccine-administered groups than in the control group, when the challenge test was performed 7 weeks after administration of the vaccine.

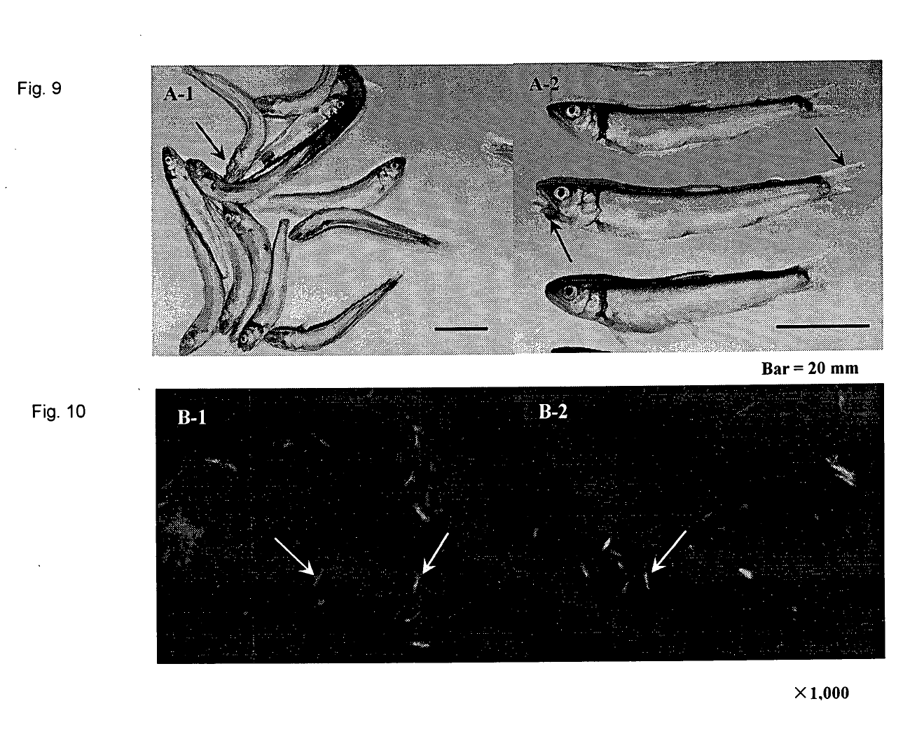

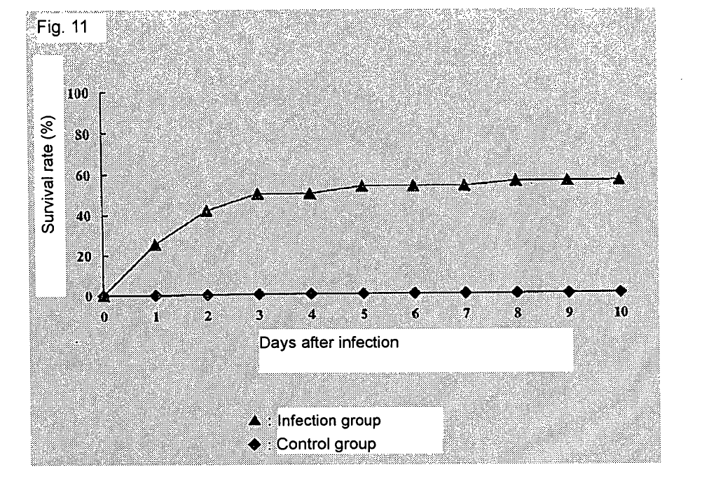

In the test fish that died in the test period of the present invention, it was confirmed whether the death was ascribed to the bacteria of the present invention or not. As shown in FIGS. 9 and 10, typical symptoms of the cold-water disease were observed in all the dead fish. It was also revealed that the cause of death of the test fish during the test period of the present invention was infection with the bacteria of the present invention, since staining of the dead fish with a fluorescent antibody was positive with respect to all the individuals tested.

### Example 6

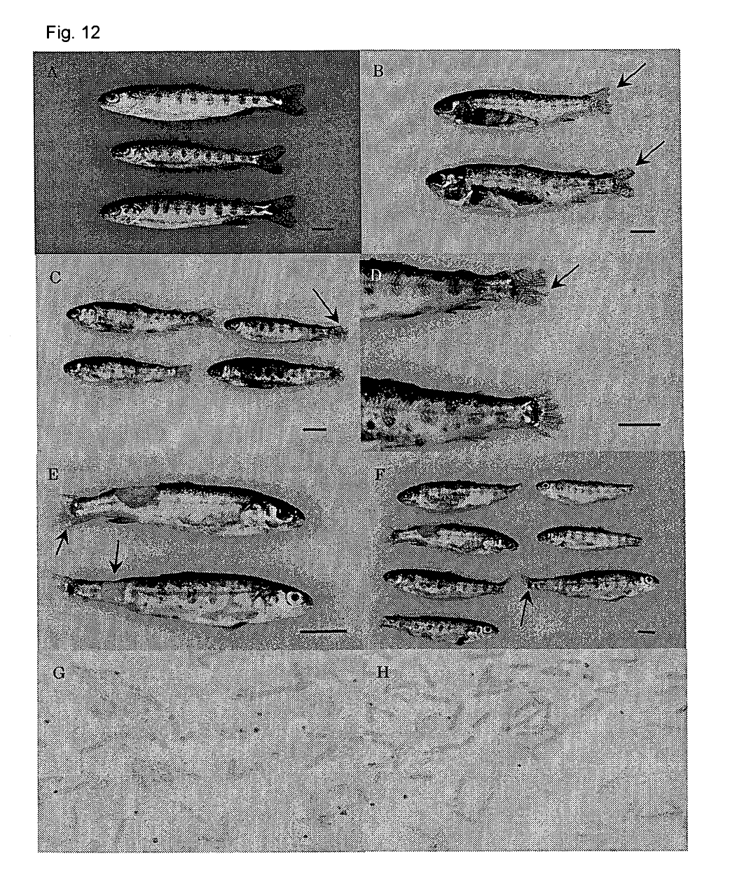

Flavobacterium psychrophilium NCMB 1947 was cultured with shaking in MCYT culture medium at 15° C., and the culture medium in the logarithmic growth phase was used for artificial infection when OD600 during 24 to 48 hours' cultivation reached 0.2 to 0.7. The bacteria of the present invention in the logarithmic growth phase were added to an aquarium of rainbow trout so that the concentration of the bacteria was 10⁶ to 10⁸ CFU/ml to attempt artificial infection by the immersion method. The body weight of rainbow trout used for the experiment was in the range of 1 to 4 g, and the water temperature was 15° C. As shown in FIG. 11, the mortality rate of the fish in the group infected with the bacteria of the invention in the logarithmic growth phase was 55.8%, in contrast to 0% in the control group (non-infection group). This result is the first successful artificial infection of rainbow trout by the immersion method. The photographs in FIG. 12 show healthy rainbow trout, symptoms of rainbow trout that died on day 1 (B, C and D) and on day 5 (E and F), and Flavobacterium psychrophilium found in the caudal fins of dead rainbow trout (G and H).

What is claimed is:

1. A vaccine against the cold-water disease in fish, comprising, as an effective component, inactivated cells of Flavobacterium psychrophilium in a logarithmic growth phase or components of the cells.

2. A vaccine composition for the cold-water disease in fish, comprising inactivated cells of Flavobacterium psychrophilium in a logarithmic growth phase or components of the cells.

3. A method for preventing the cold-water disease in fish, comprising administering an effective dosage of inactivated cells of Flavobacterium psychrophilium in a logarithmic growth phase or components of the cells.
